(19) United States
(12) Patent Application Publication
(10) Pub. No.: US 2007/0105931 A1
(43) Pub. Date: May 10, 2007

(54) Methods and compositions for inhibiting the proliferation of prostate cancer cells

(75) Inventor: Charles Y.F. Young, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research

(21) Appl. No.: 10/567,477

(22) PCT Filed: Aug. 4, 2004
(86) PCT No.: PCT/US04/25336

§ 371 Date: Sep. 11, 2006

Related U.S. Application Data
(60) Provisional application No. 60/492,367, filed on Aug. 4, 2003.

(51) Int. Cl. A61K 31/415; A61K 31/165; C12Q 1/68

(52) U.S. Cl. 514/406; 514/618; 435/6

(57) ABSTRACT

The invention provides for methods of monitoring the proliferation of cultured prostate cancer cells in the presence of celecoxib and/or nimesulide, methods of treating an individual with prostate cancer or at risk of developing prostate cancer, and methods of reducing the risk of recurrence of prostate cancer in an individual who had previously been treated for prostate cancer. Methods of the invention further include treating an individual with benign prostatic hyperplasia (BPH) with celecoxib and/or nimesulide as well as methods of screening for compounds that inhibit the proliferation of prostate cancer cells. The invention provides for compositions and articles of manufacture containing celecoxib and/or nimesulide in particular formulations, and celecoxib and/or nimesulide with a second compound that also exerts an effect on the androgen receptor.

### CROSS REFERENCE TO RELATED APPLICATIONS

This application claims priority under 35 U.S.C. §119(e) of U.S. Application No. 60/492,367, filed Aug. 4, 2003.

### TECHNICAL FIELD

This invention relates to prostate cancer, and more particularly to methods and compositions for inhibiting the proliferation of prostate cancer cells.

### BACKGROUND

The prostate gland is located between the bladder and the rectum and wraps around the urethra. The prostate is composed of glandular tissue that produces a milky fluid and smooth muscles that contract during sex and squeeze this fluid into the urethra where it mixes with other fluid and sperm to form semen. The prostate gland converts testosterone to a more powerful male hormone, dihydrotestosterone, which affects the size of the gland and plays an important role in prostate cancer.

Prostate cancer is a malignant tumor that arises in the prostate gland and can eventually spread through the blood and lymph fluid to other organs, bones, and tissues. Prostate cancer is the most commonly diagnosed cancer in the U.S., and it is the second leading cause of cancer death in American men after non-melanoma skin cancer. Although prostate cancer is just as common in Japan as in the United States, death rates from prostate cancer are significantly lower in Japan. It is unlikely that these differences are all genetic, because Japanese men who migrate to the United States die of prostate cancer with increasing frequency as a function of the number of years they reside in the United States. It is possible that this paradox could be explained, at least in part, by dietary factors.

Benign prostatic hyperplasia (BPH) is a benign enlargement of the prostate gland caused by the growth of both glandular and stromal tissues. Because the prostate enlargement in BPH is affected by testosterone, many men are concerned that it may be related to prostate cancer. A ten-year study, however, found no higher risk for prostate cancer in men with or that have experienced BPH. BPH develops in the inner zone of the prostate (i.e., predominantly stromal cells), while cancer tends to develop in the outer area (i.e., epidermal cells).

### SUMMARY

It is reported herein that the expression, the transactivating ability, and/or the IL6-mediated activation of the androgen receptor was inhibited by one or more NSAIDs. Accordingly, the invention provides for methods of monitoring the proliferation of cultured prostate cancer cells, methods of treating an individual with prostate cancer or at risk of developing prostate cancer, and methods of reducing the risk of recurrence of prostate cancer in an individual who had previously been treated for prostate cancer. The invention further includes methods of treating an individual with benign prostatic hyperplasia (BPH) or at risk of developing BPH as well as methods of screening for compounds that inhibit the proliferation of prostate cancer cells. The invention provides for compositions and articles of manufacture containing one or more NSAIDs in particular formulations, or one or more NSAIDs with a second compound that also exerts an effect on the androgen receptor.

In one aspect, the invention provides methods of monitoring the proliferation of cultured prostate cancer cells in the presence of one or more NSAIDs. Such a method includes contacting the prostate cancer cells with one or more NSAIDs; and determining the level of expression, the transactivating ability, and/or the IL6-mediated activation of an androgen receptor. Generally, a decrease in the expression, the transactivating ability, and/or the IL6-mediated activation of the androgen receptor indicates an inhibitory effect by the NSAID on the proliferation of the prostate cancer cells.

In another aspect, the invention provides for methods of screening for compounds that inhibit the proliferation of prostate cancer cells. Such a method includes contacting prostate cancer cells with a compound; and determining the level of expression, the transactivating ability, and/or the IL6-mediated activation of an androgen receptor. Generally, decreased expression, transactivating ability, and/or IL6-mediated activation of the androgen receptor in the prostate cancer cells compared to prostate cancer cells not contacted with the compound indicates a compound that inhibits the proliferation of prostate cancer cells.

In another aspect, the invention provides methods of treating an individual with prostate cancer or at risk of developing prostate cancer. Such a method includes identifying an individual with prostate cancer or at risk of developing prostate cancer; and administering a dose of one or more NSAID to the individual in an amount effective to inhibit expression, transactivating ability, and/or IL6-mediated activation of an androgen receptor. Generally, an inhibition of the expression, the transactivating ability, and/or the IL6-mediated activation of the androgen receptor inhibits the proliferation of prostate cancer cells, thereby treating the individual.

In yet another aspect, the invention provides methods of reducing the risk of recurrence of prostate cancer in an individual, wherein the individual previously had been treated for prostate cancer. Such a method includes administering a dose of one or more NSAIDs to the individual in an amount effective to inhibit expression, transactivating ability, and/or IL6-mediated activation of an androgen receptor. Generally, inhibiting the expression, the transactivating ability, and/or the IL6-mediated activation of the androgen receptor inhibits the proliferation of prostate cancer cells, thereby reducing the risk of recurrence of prostate cancer in the individual. In some embodiments, the previous treatment for prostate cancer in the individual included a radical prostectomy.

In still another aspect, the invention provides methods of treating an individual with benign prostatic hyperplasia (BPH) or at risk of developing BPH. Such a method includes identifying an individual with BPH; and administering a dose of one or more NSAIDs to the individual in an amount effective to inhibit expression, transactivating ability, and/or IL6-mediated activation of an androgen receptor, thereby treating the individual.

In other aspects, the above-described methods can further include monitoring the expression, the transactivating ability, and/or the IL6-mediated activation of the androgen receptor in the individual; monitoring the individual for a dose-dependent reduction in prostate-specific antigen (PSA) levels, and/or monitoring the individual for a reduction in human glandular kallikrein (hK2) levels. Typically, a dose-dependent reduction in PSA correlates with a dose-dependent decrease in the expression, the transactivating ability, and/or the IL6-mediated activation of the androgen receptor, while a reduction in hK2 correlates with a decrease in the expression, the transactivating ability, and/or the IL6-mediated activation of the androgen receptor. In certain embodiments, the dose of the one or more NSAIDs can be adjusted, if necessary, to achieve or maintain the dose-dependent reduction in PSA or the reduction in hK2.

In some embodiments of the above-described methods, the effective dose can be from about 10 mg/kg to about 300 mg/kg. A representative individual is a human, and representative routes of administration include orally, transdermally, intravenously, intraperitoneally, or using an implant. Representative NSAIDs include celecoxib and/or nimesulide.

In another aspect, the invention provides compositions that include one or more NSAIDs, one or more compounds that inhibits expression of a gene encoding an androgen receptor, inhibits nuclear localization of an androgen receptor, and inhibits the transactivating ability of an androgen receptor, and a pharmaceutically acceptable carrier. Representative NSAIDs include celecoxib and/or nimesulide, and representative compounds include silymarin, silibin, docosahexaenoic acid (DHA), eicosapentaenoic acid (EPA), quercetin, perillyl alcohol (POH) or a derivative thereof, resveratrol, flufenamic acid, tea polyphenols, and anti-androgen compounds.

In still another aspect, the invention provides for compositions that include one or more NSAIDs formulated for transdermal delivery to the prostate of an individual or formulated for implantation near the prostate of an individual. Typically, delivery to the prostate inhibits the expression, the transactivating ability, and/or the IL6-mediated activation of the androgen receptor. Representative NSAIDs include celecoxib and/or nimesulide.

In yet another aspect, the invention provides for articles of manufacture that include the above-described compositions and packaging material. Generally, the packaging material includes instructions for using the composition to inhibit expression, transactivating ability, and/or IL6-mediated activation of an androgen receptor in an individual. Articles of manufacture of the invention can further include compositions for monitoring the expression, the transactivation, and/or the IL6-mediated activation of the androgen receptor; compositions for monitoring PSA; and/or compositions for monitoring hK2.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is a graph showing the effects of NSAIDs on the expression of PSA and hK2 proteins in prostate cancer cells ±1 nM Mib. LNCaP cells (Panel A) and LAPC-4 cells (Panel B) were treated with the indicated concentrations of celecoxib or nimesulide for 7 days. PSA and hK2 values were normalized to growth response measured by a 3-(4,5-dimethylthiazol-2-yl)-2,5-diphenyltetrazolium bromide assay and expressed as a percentage of that group treated with Mib only. Error bars indicate the SE of four separate experiments.

FIG. 2 is a graph showing LNCaP cells transected with a luciferase reporter plasmid that contains the 6-kb PSA promoter or three copies of ARE or control plasmid (pGL3) and a CMV-β-gal expression vector and treated with NSAIDs ±1 nm Mib for 24 h. *, P<0.05 for PSA promoter and hK2-3ARE promoter. After normalization with β-gal, luciferase activities were expressed as a percentage of that of groups treated with Mib only.

FIG. 3A is a graph showing LNCaP cells co-transfected with AR promoter-luciferase reporter (AR-pGL3) or the parental vector (pGL3) and CMV-β-gal and treated with 1 nm Mib and NSAIDs at the indicated concentrations for 24 h. FIG. 3B is a graph showing LNCaP cells contransfected with AR promoter (−74/+87)-pGL3, AR promoter (−1380/+577)-pGL3, or the parental vector (pGL3) plus CMV-β-gal and different amounts of c-jun expression vector for 24 h. The resulting activities of both AR-pGL3 were further normalized to β-gal and expressed as a percentage of the AR promoter (−1380/+577) without NSAIDs or c-jun *, P<0.05 for AR promoter (−1380/+577)-pGL3; **, P<0.05 for AR promoter (=74/+87)-pGL3.

FIG. 4 are graphs demonstrating that IL6 activates the androgen receptor, and that the IL6-mediated activation of the androgen receptor is inhibited by celecoxib and nimesulide.

FIG. 5 are graphs demonstrating that in the presence of IL6, celecoxib and nimesulide inhibited STAT3-mediated expression of a reporter gene.

### DETAILED DESCRIPTION

It is reported herein that the expression, the transactivating ability, and/or the IL6-mediated activation of the androgen receptor was inhibited by NSAIDs. It was shown herein that one or more NSAIDs inhibited androgen-stimulated secretion of both prostate-specific antigen (PSA) and hK2. Expression, transactivating ability, and/or I16-mediated activation of the androgen receptor was diminished by treatment with one or more NSAIDs. The invention provides a novel aspect of NSAIDs in that NSAIDs can reduce androgen receptor expression, attenuate androgen receptor-mediated transactivation of prostate cancer-specific genes in androgen-responsive prostate cancer cells, and/or attenuate IL6-mediated activation of the androgen receptor. Thus, the invention provides for methods of preventing or treating prostate cancer using one or more NSAIDs.

The Androgen Receptor and Prostate Cancer

Androgens play an important role in the proliferation, differentiation, maintenance, and function of the prostate. The androgen receptor is the essential mediator for androgen action and is a ligand-dependent transcription factor belonging to the nuclear steroid hormone receptor superfamily. Androgens can enhance androgen receptor protein levels by increasing the half-life, as well as by stimulating the phosphorylation of the androgen receptor. Phosphorylation may affect numerous characteristics of nuclear receptors including ligand binding, nuclear translocation, dimerization, DNA binding, and protein-protein interactions.

Evidence shows that androgens are also involved in the development and progression of prostate cancer. Therefore, the androgen receptor also plays a critical role in the development of prostate cancer, in part due to overstimulation of the receptor by androgens. Prostate cancer also has been attributed to altered transactivation activities of the receptor or to mutations in the androgen receptor that, for example, enable the receptor to respond to non-androgen steroids. The androgen receptor can be expressed in all stages of prostate cancer, and at least one-third of advanced prostate cancers contain amplified androgen receptor genes.

The utilization of androgen deprivation as a treatment for advanced prostate cancer was first demonstrated in 1941 and has become a standard treatment. Based on the morbidity associated with ablation of the adrenal glands, castration alone was the gold standard until the 1980s, when anti-androgen agents, including cyproterone acetate, megestrol acetate, and flutamide, were developed to compete with androgen for binding to the androgen receptor. Many new classes of drugs that interfere with androgen production and function have been identified.

In spite of the apparent regression of tumors by hormone therapy, however, prostate cancer often recurs within 3 years and becomes hormone refractory with a potentially fatal outcome. Many molecular mechanisms have been postulated to be responsible for the development of recurrent hormone-refractory tumors with most involving alterations in the function of the androgen receptor and its complex signaling pathways. The androgen receptor can be activated by a number of growth factors or cytokines in the absence of androgens, or by low levels of androgens or other non-androgenic steroid hormones after hormone therapy. The majority of hormone-refractory cancers still express the androgen-responsive prostate-specific antigen (PSA). PSA is a protein secreted by the epithelial cells of the prostate gland, including prostate cancer cells. An abnormally high level of PSA is indicative of abnormal prostate cells. The presence of PSA indicates that the androgen receptor signaling pathway is functional.

Nucleic acid sequences encoding androgen receptors have been cloned and sequenced from numerous organisms. Representative organisms and GenBank accession numbers for androgen receptor sequences therefrom include the following: frog (Xenopus laevis, U67129), mouse (Mus musculus, 109558), rat (Rattus norvegicus, 292896), human (Homo sapiens, 105325), rabbit (Oryctolagus cuniculus 577829), cow (Bos taurus, Z75313, Z75314, Z75315), canary (Serinus canaria, 414734), and whiptail lizard (Cnemidophous uniparens, 1195596). Additionally, Cancer Genetics Web (cancer-genetics.org on the World Wide Web) contains database entries for wild-type and mutant androgen receptor sequences.

Prostate cancer cells can be identified using several criteria. Prostate cancer cells in culture (e.g., LNCaP cells) can be characterized by the response of such cells to androgens or to androgenic agonists or antagonists. Molecular markers, such as increased or decreased expression of androgen-regulated genes or genes involved in prostate cancer (e.g., PSA, hK2, c-jun, ODC, and NKX3.1) also can be used to characterize prostate cancer cells in culture. Prostate cancer in vivo can be identified by a digital rectal examination of a patient, or by imaging or scanning techniques (e.g., magnetic resonance imaging (MRI), or prostascint scans). In addition, the degree of cellular differentiation can be evaluated in prostate cancer cells from an individual, typically removed via a biopsy of prostate tissue, using a Gleason score. Further, there are several commercially available diagnostic tests for PSA and PSA-II (e.g., Roche Diagnostics Inc., Indianapolis, Ind.) to screen individuals for prostate cancer and to monitor individuals undergoing treatment for prostate cancer. Prostate cancer can be staged, for example, using a Partin Table and/or a Partin II Table (see Partin et al., 1994, Urology, 43:649-59 and theraseed.com/gloss on the World Wide Web for more information).

Methods of Monitoring and Inhibiting the Proliferation of Prostate Cancer Cells

The invention provides for methods of monitoring the proliferation of prostate cancer cells. According to the methods of the invention, the proliferation of prostate cancer cells can be monitored by contacting those cells with one or more NSAIDs and then determining the expression, the transactivating ability, and/or the IL6-mediated activation of the androgen receptor using conventional methods (e.g., methods described herein). A decrease in the expression, the transactivating ability, and/or the IL6-mediated activation is indicative of an inhibitory effect by the NSAID(s) on the proliferation of the prostate cancer cells.

Proliferation of prostate cancer cells as used herein refers to an increase in the number of prostate cancer cells (in vitro or in vivo) over a given period of time (e.g., hours, days, weeks, or months). It is noted that the number of prostate cancer cells is not static and reflects both the number of cells undergoing cell division and the number of cells dying (e.g., by apoptosis). An inhibition of the proliferation of prostate cancer cells can be defined as a decrease in the rate of increase in prostate cancer cell number, a complete loss of prostate cancer cells, or any variation therebetween. With respect to tumors, a decrease in the size of a tumor can be an indication of an inhibition of proliferation.

Prostate cancer cells that can be maintained in culture and are useful in the invention include without limitation LNCaP cells and LAPC-4 cells. The LNCaP cell line is an established androgen-responsive prostate cancer cell line obtained from a lymph node metastasis of a prostate cancer patient. LNCaP cells express the androgen receptor and a number of androgen-inducible genes such as PSA, human glandular kallikrein (hK2), NKX3.1 and ornithine decarboxylase (ODC). The gene encoding the androgen receptor in the LNCaP cell line contains a mutation in its ligand-binding domain, but otherwise is functional. LAPC-4 cells, another androgen responsive prostate cancer cell line suitable for use in the invention, expresses a wild-type androgen receptor. LAPC-4 cells additionally express PSA and hK2, which are up-regulated in the LAPC-4 cells by androgens. Other prostate cancer cell lines are available and include PC-3 and DU145.

The invention further provides for methods of treating an individual with prostate cancer or at risk of developing prostate cancer. An individual is first identified as having prostate cancer or being at risk for developing prostate cancer and is then administered an effective dose of one or more NSAIDs. The expression, the transactivating ability, and/or the IL6-mediated activation of the androgen receptor can be monitored in the individual to evaluate the effects of one or more NSAIDs on prostate cancer cells. Generally, an inhibition of the expression, the transactivating ability, and/or the IL6-mediated activation of the androgen receptor by the NSAID(s) inhibits the proliferation of prostate cancer cells, thereby treating the individual.

Non-steroidal anti-inflammatory drugs (NSAIDs) generally are drugs that have pain-relieving (analgesic) and inflammation-reducing effects. NSAIDs work primarily by preventing the formation of prostaglandins, which are produced by both COX-1 and COX-2. Traditional NSAIDs (e.g., aspirin, ibuprofen, and naproxen) inhibit both COX-1 and COX-2. Newer NSAIDs (e.g., celecoxib, diclofenac, etodolac, fenoprofen, indomethacin, ketoprofen, ketoralac, nabumetone, oxaprozin, sulindac, tolmetin, and rofecoxib) selectively inhibit COX-2, are effective for treatment of musculoskeletal pain, and lack many of the side effects associated with traditional NSAIDs. Other classes of NSAIDs also have been identified. For example, nimesulide has weak inhibitory action against COX-2, but has potent anti-inflammatory activity. Nimesulide behaves as a competitive inhibitor of histamine release and hence possesses anti-histaminic and anti-allergic properties. Any of a number of NSAIDs or combinations thereof can be used in the methods of the invention.

For the purpose of this invention, the NSAID(s) can be administered orally, transdermally, intravenously, intraperitoneally, or by implantation. The route of administration typically depends on a variety of factors, such as treatment environment and therapeutic goals. Administration of the NSAID(s) can be on a continuous or an intermittent basis. A continuous administration can be, for example, five times a day, once a day, once every other day, once a week, or once a month. In addition, preparations for administration of the NSAID(s) can be suitably formulated to give controlled release of the compound. Preparations for intravenous and intraperitoneal administration can include sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, as well as alcohol, saline, and buffered solutions. Other additives such as, for example, antimicrobials, anti-oxidants, chelating agents, inert gases, steroids, anti-inflammatory agents, immunosuppressants, vasodilators, vasoconstrictors, and the like may also be present.

Tablets or capsules for oral administration can be prepared by conventional means with pharmaceutically acceptable excipients such as binding agents (e.g. pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Tablets can be coated by methods known in the art. Liquid preparations for oral administration can take the form of, for example, solutions, syrups or suspension, or they can be presented as a dry product for constitution with saline or other suitable liquid vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl- or propyl-p-hydroxybenzoates or sorbic acid). The preparations can also contain buffer salts, flavoring, coloring and sweetening agents as appropriate.

Preparations for transdermal administration are known in the art. Such transdermal preparations can be in the form of a scrotum patch or a patch for application on the back, abdomen, thighs, or buttocks. A transdermal patch typically includes a soft flexible backing (e.g., polyester or polyester/ethylene-vinyl acetate copolymer), a reservoir (in some cases, the compound or composition, e.g. one or more NSAIDs, can be deposited as a film on the ethylene-vinyl acetate copolymer or can be combined with, for example, alcohol and a gelling agent such as hydroxypropyl cellulose), and an adhesive backing made out of, for example, polyisobutylene and colloidal silicon dioxide (usually with a removable liner (e.g., silicone-coated polyester, or fluorocarbon diacrylate) to protect the adhesive until the patch is applied). A transdermal patch also can contain a formulation (e.g., polyisobutylene adhesive) to control the rate of release of the compound or composition.

Implantable devices are known in the art and can be in the form of a pellet or a seed containing or coated with a compound or composition, e.g., one or more NSAIDs. A pellet or seed can be a metal alloy (e.g., cobalt, or palladium) or an inert plastic or other substance. A device for implantation in or near the prostate can be delivered using a delivery catheter (similar to brachytherapy) and can be deposited in or near the prostate transperineally, transrectally, or transurethrally. A transrectal ultrasound can be used in conjunction with implantation to visualize and image the prostate and the positioning of the implantable device.

According to the invention, an effective dose of the NSAID(S) is an amount that inhibits the expression, the transactivating ability, and/or the IL6-mediated activation of the androgen receptor, thereby inhibiting the proliferation of prostate cancer cells. Inhibition of the expression, the transactivating ability, and/or the IL6-mediated activation of the androgen receptor and the subsequent inhibition of the proliferation of prostate cancer cells can be determined using methods and assays described herein. It is anticipated that an effective dose of the NSAID(s) is from about 10 mg of NSAIDs per kg weight of the individual (mg/kg) to about 300 mg/kg. Toxicity and therapeutic efficacy of different doses of the NSAID(s) can be determined by standard pharmaceutical procedures in cell cultures or experimental animals, e.g. by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio of LD₅₀/ED₅₀. Doses of the NSAID(s) that exhibit high therapeutic indeces are preferred. An effective dose of the NSAID(s) can be delivered in a single dose or as multiple doses over a period of time.

The transactivating ability of the androgen receptor can be examined by evaluating the expression of genes whose transcription is regulated by androgen receptor binding. Such genes include PSA, h2k, NKX3.1, and ODC. The amount of transcript and/or protein of such genes in the presence and absence of the compound can be readily determined using art-routine methods such as those described herein. Alternatively, prostate cancer cells in culture can be made transgenic for one or more androgen-regulated genes and the expression of such transgenes can be evaluated in the presence and absence of a compound.

In addition, the invention provides methods of reducing the risk of recurrence of prostate cancer in an individual that previously had undergone treatment for prostate cancer. Such methods include administering an effective dose of one or more NSAIDs to the individual such that the expression, the transactivating ability, and/or the IL6-mediated activation of the androgen receptor is inhibited. Inhibiting the expression, the transactivating ability, and/or the IL6-mediated activation of the androgen receptor inhibits the proliferation, and therefore the recurrence, of prostate cancer cells. Treatments for prostate cancer that an individual might undergo include hormone therapy, chemotherapy, radiation therapy, and, oftentimes, a prostatectomy, in which part or all of the prostate gland is removed. A radical prostatectomy includes removal of the entire prostate as well as the seminal vesicles. Due to a high incidence of prostate cancer recurring even following such treatments (including a radical prostatectomy), methods of the invention provide for administration of one or more NSAIDs during or following such treatments. Administration of the NSAID(s) may be particularly useful following a radical prostatectomy.

The invention additionally provides for a method of treating an individual with benign prostatic hyperplasia (BPH). Individuals with BPH may present with prostatitis and/or difficulty urinating, and an enlarged prostate due to BPH is typically palpable during a digital rectal exam. Methods of the invention include identifying an individual with BPH, and administering a dose of one or more NSAIDs to the individual in an amount effective to inhibit the expression, the transactivating ability, and/or the IL6-mediated activation of an androgen receptor. Such an inhibition of the expression, the transactivating ability, and/or the IL6-mediated activation reduces the androgen receptor-mediated growth response and thereby treats the individual with BPH.

Methods of Screening Compounds

The invention provides for methods of screening for compounds that inhibit the proliferation of prostate cancer cells by decreasing the expression, the transactivating ability, and/or the IL6-mediated activation of the androgen receptor. Screening methods are one of the fundamental tools used in molecular biology for rapid and efficient identification and evaluation of compounds. Screening methods of the invention include contacting prostate cancer cells with a compound under conditions and for a time sufficient to allow the compound to enter the cell, and determining the level of expression, the transactivating ability, and/or the IL6-mediated activation of the androgen receptor. Generally, decreased expression, transactivating ability, and/or IL6-mediated activation of the androgen receptor in cells compared to cells not contacted with the compound indicates a compound that inhibits the proliferation of prostate cancer cells. Such compounds can be evaluated using prostate cancer cells in culture, such as LNCaP or LAPC-4 cells, or can be evaluated using a cell-free system.

Methods of evaluating the transactivating ability of the androgen receptor are described above. Expression of a gene encoding an androgen receptor in prostate cancer cells can be examined in the presence and absence of a compound using Northern blot analysis (to evaluate transcription) and/or Western blot analysis (to evaluate translation). Techniques to isolate RNAs and proteins from cells as well as methods of separation (e.g., electrophoretically) are well known and routine in the art. Androgen receptor mRNA can be detected by hybridization with a labeled oligonucleotide probe that is complementary to a portion of the androgen receptor transcript. Androgen receptor proteins can be detected by contacting proteins from a cell with a labeled agent that selectively binds to the androgen receptor protein. Conditions for allowing and detecting hybridization of nucleic acids or binding of antibodies to proteins are well known in the art. Antibodies that have binding affinity to androgen receptor proteins are commercially available (e.g., from Research Diagnostics Inc. (Flanders, N.J.) and Alpha Diagnostic International (San Antonio, Tex.)). The term “label”, with regard to an oligonucleotide probe or an antibody is intended to encompass direct labeling of the oligonucleotide or antibody by coupling a detectable substance to the oligonucleotide or antibody, as well as indirect labeling of the oligonucleotide or antibody by reactivity with a detectable substance. Examples of labels and detectable substances are well known in the art. Additional methods to detect androgen receptor MRNA (e.g., RT-PCR or dot blots) or protein (e.g., immunoassays or chromatography) are well known and also practiced routinely in the art.

The ability of the androgen receptor to translocate to the nucleus also can be evaluated in the presence and absence of a compound to determine if the compound inhibits the nuclear localization of the androgen receptor. Nuclei are typically isolated using an appropriate gradient such as a sucrose gradient, a percol gradient, or the like. The nuclei can be lysed (for example, by exposure to sonication, or ultrasound waves) and androgen receptor protein can be detected using routine methods such as Western blotting. Nuclear translocation also can be examined using, for example, immunocytochemistry to identify androgen receptor protein in the nucleus and/or outside of the nucleus.

In addition, the amount of c-jun protein can be evaluated as an indicator of androgen receptor activity. When overexpressed, c-jun has been shown to inhibit the transactivating ability of the androgen receptor, c-jun is a partner with c-fos in the transcription factor AP-1. Increased evidence suggests that the function of the androgen receptor may be affected by an interaction with AP-1.

Compositions and Articles of Manufacture

The invention provides compositions that include one or more NSAIDs and at least one other compound selected for its particular mechanism of action on the androgen receptor. The mechanism of action exerted by the other compound(s) can be one or more of the following: inhibition of the expression of a gene encoding an androgen receptor; inhibition of the nuclear localization of an androgen receptor; or inhibition of the transactivating ability of an androgen receptor. Representative compounds exhibiting such mechanisms of action include the following: resveratrol, perillyl alcohol (POH) or a derivative thereof, and omega-3 fatty acids (transactivating ability); silymarin (nuclear localization); flufenamic acid, tea polyphenols (e.g., (−)-epigallocatechin gallate (EGCG)), and quercetin (expression); and numerous anti-androgen compounds (e.g., bicalutamide, flutamide, nilutamide, or cyproterone).

Compositions containing one or more NSAIDs can be formulated for delivery to the prostate. In one aspect, the NSAID(s) are formulated for transdermal delivery to the prostate. In another aspect, compositions containing the NSAID(s) can be formulated for implantation in or near the prostate. Delivery of compositions containing the NSAID(s) directly to the prostate of an individual inhibits the expression, the transactivating ability, and/or the IL6-mediated activation of the androgen receptor. Formulations for administration of the NSAID(s) are described above and also apply to the disclosed compositions containing one or more NSAIDs.

A composition containing the NSAID(s) can be in any form provided the composition can be administered to an individual in an amount and for a duration effective to inhibit the expression, the transactivating ability, and/or the IL6-mediated activation of the androgen receptor gene, thereby inhibiting the proliferation of prostate cancer cells. Pharmaceutically acceptable carriers include solvents, dispersion media, coatings, antibacterial and anti-fungal agents, isotonic and absorption delaying agents and the like, appropriate to specific routes of administration.

NSAID compositions of the invention that are effective for inhibiting the expression, the transactivating ability, and/or the IL6-mediated activation of the androgen receptor as described herein can be combined with packaging material and sold as a kit (i.e., an article of manufacture). Components and methods for producing articles of manufactures are well known. In addition to an NSAID composition, articles of manufacture can include oligonucleotide probes, antibodies, and/or other useful agents for determining the expression, the transactivating ability, and/or the IL6-mediated activation of the androgen receptor. Instructions describing how the composition can be used for inhibiting the expression, the transactivating ability, and/or the IL6-mediated activation of the androgen receptor to thereby inhibit the proliferation of prostate cancer cells can be included in such kits.

In accordance with the present invention, there may be employed conventional molecular biology, microbiology, biochemical and recombinant DNA techniques within the skill of the art. Such techniques are explained fully in the literature. The invention will be further described in the following examples, which do not limit the scope of the invention described in the claims.

### EXAMPLES

### Example 1

### Cell Culture

Human prostate cancer cell lines LNCaP (American Type Culture Collection, Manassas, Va.) and LAPC-4 (kindly provided by Dr. Charles L. Sawyers; Zhu et al., 1999, Endocrinology, 140:5451-4) were maintained in RPMI 1640 (Mediatech, Hercules, Calif.) containing 5% FBS (Biofluids, Rockville, Md.) at 37° C. and 5% CO₂. To avoid potential interference of existing steroids in FBS, the media were first replaced by serum-free RPMI 1640 for 24 h. Cells were then cultured in RPMI 1640 with 5% charcoalstripped FBS supplemented with or without 1 nM Mib (New England Nuclear, Boston, Mass.), a nomnetabolizable, synthetic androgen.

### Example 2

### Growth Response and PSA and hK2 Levels

Cells were plated in 24-well plates at 2×10⁴ cells/well. Forty-eight h after plating, cells were treated with celecoxib or nimesulide (LKT Lab, St. Paul, Minn.) and other NSAIDs as shown in Table 1 at different doses in the presence or absence of Mib. MTS assay (Promega, Madison, Wis.) was performed to determine cell proliferation 6 days after the treatment. To measure secreted PSA and hK2 levels, 400 μl of spent medium from cells treated for 6 days were collected. PSA and hK2 proteins levels were determined using specific immunoassays (Mayo Immunochemical Core Facility). These measurements were used to the calculate 50% inhibitory concentration (IC50) of each of the NSAIDs.

TABLE 1
Effects of selected NSAIDs on growth responses and expression of androgen-regulated
genes in androgen-responsive human prostate cancer cell lines

| | Selective | LNCaP (IC₅₀)^{a} | | | LAPC-4(IC₅₀)^{a} | | |
|---|---|---|---|---|---|---|---|
| NSAIDs | inhibitor to | Growth | PSA | hK2 | Growth | PSA | hK2 |
| Aspirin | COX-1 and -2 | >1000 | >1000 | >1000 | >1000 | >1000 | >1000 |
| Ibuprofen | COX-1 and -2 | >1000 | 783.3 | 860 | 740 | 870 | 803 |
| Meloxicam | COX-2 | 193.1 | 300 | 377 | 92 | >300 | >300 |
| Ketoprofen | COX-1 and -2 | >300 | >300 | >300 | >300 | >300 | >300 |
| Flurbiprofen | Cox-1 and -2 | >300 | 206 | 281 | 347 | 234 | 221 |
| Nimesulide | COX-2 | 38.2 | 27 | 23 | 104 | 76.5 | 61 |
| Sulindac | COX-1 and -2 | >300 | >300 | >300 | >300 | >300 | >300 |
| Sulindac sulfone | b | >3001 | 206 | 97.6 | 253 | 193.8 | >300 |
| Celecoxib | COX-2 | 32.6 | 20 | 29.6 | 44.5 | 28.7 | 43.7 |
| Fenoprofen | COX-1 and -2 | >300 | 210.4 | 228.6 | N/D^{c} | N/D | N/D |
| Indomethacin | COX-1 and -2 | >300 | 224 | 272 | 170 | 203 | 207 |

### Example 3

### Western Blot Analysis

Cells were seeded at 1×10⁵ cells/plate in 100 mm dishes. Cells grown in log phase were co-treated with 1 nM Mib and different concentrations of celecoxib or nimesulide for 15 or 24 h. The cells were collected by centrifugation and washed with nimesulide for 15 or 24 h. The cells were collected by centrifugation and washed with cold PBS. Cell lysates were prepared in radioimmunoprecipitation assay buffer (PBS containing 1% NP40, 0.5% sodium deoxycholate, 0.1% SDS plus freshly added protease inhibitors, 100 μg/ml phenylmethylsulfonyl fluoride, 30 μl/ml aprotinin, and 1 mM sodium orthovanadate) and used for Western blot analysis. The sample filters were immunoblotted with c-Jun, phospho-c-Jun (Cell Signaling, Beverly, Mass.), AR (PharMingen, San Diego, Calif.), and FKBP51 (a gift from Dr. D. O. Toft; Mayo Clinic) specific primary antibodies and horseradish peroxidase-conjugated secondary antibodies and visualized by enhanced chemiluminescence (Amersham Pharmacia, Piscataway, N.J.).

### Example 4

### Transfections and Transcriptional Reporter Assays

LNCaP cells were plated into 60-mm dishes. Cells at 50-70% confluence were transfected with the appropriate constructs [6-kb PSA promoter-pGL3, AR promoter (−74/+87)-pGL3, AR promoter (−1380/+577)-pGL3, hK2 3xARE-SV40 minimal promoter pGL3, or empty pGL3 vectors] by using the method described previously (Ren et al., 2000, Oncogene, 19:1924-32). Twenty-four h after transfection, cells were treated with celecoxib or nimesulide in combination with Mib or ethanol. Whole cell lysate was prepared for luciferase assay according to the manufacturer's instructions (Promega). CMV-β-gal expression vector was also cotransfected for normalization of transfection efficiency. Each transfection was done three times, and SDs were calculated.

### Example 5

### Statistics

The data were analyzed by Student's t test. P<0.05 was accepted as the level of significance.

### Example 6

### Celecoxib and Nimesulide Inhibited the Expression of Androgen Up-Regulated Genes

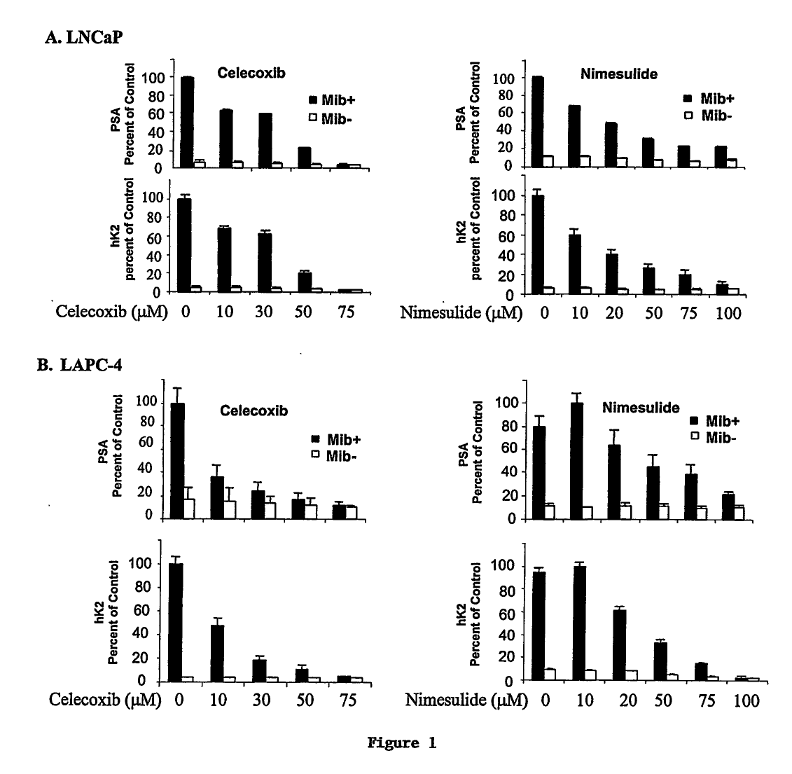

The effects of several NSAIDs on inhibition of androgen action and growth in prostate cancer cells was examined. Using PSA and hK2, two well-established AR target genes, as markers, the effects of a panel of 11 NSAIDs was tested on androgen action in two androgen-responsive human prostate cancer cell lines, LNCaP and LAPC-4, respectively. Among the NSAIDs tested, COX-2-specific inhibitors seem to have a higher potency than other NSAIDs in inhibiting androgen action. Celecoxib and nimesulide showed the lowest IC₅₀ concentrations in both cell lines (Table 1). Because of their highest potency on inhibition of cell growth and androgen function, celecoxib and nimesulide were chosen for additional studies. FIG. 1 illustrates that expression of PSA and hK2 was suppressed by celecoxib and nimesulide in a dose-dependent manner in the two cell lines. Significant inhibitory activity was observed for celecoxib at a concentration of 10 μM for both PSA and hK2. Nimesulide at 10 μM resulted in a similar inhibition of PSA, although a higher concentration was required to achieve significant down-regulation in LAPC-4 cells. Recently, it was discovered that FKBP51, an immunophilin, is up-regulated by androgens. Similarly, it was found in the experiments described herein that androgen-up-regulated FKBP51 protein expression was alleviated by celecoxib and nimesulide treatment as measured by Western blot analysis using specific antibody. These results suggest these NSAIDs are potent inhibitors of AR-induced gene expression.

### Example 7

### Celecoxib and Nimesulide Inhibited AR-induced Gene Expression and AR Promoter Activity at the Transcription Level

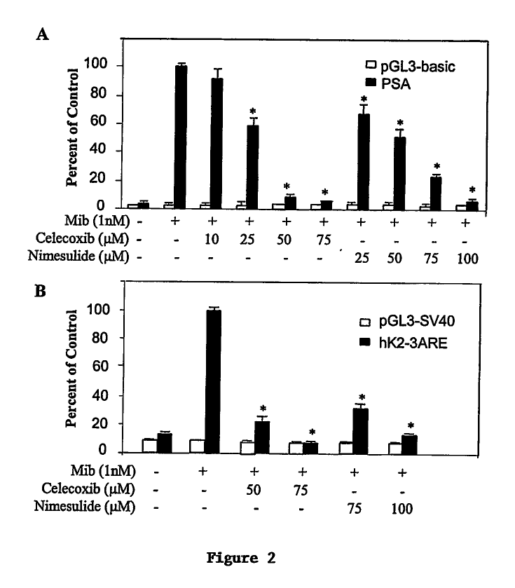

To test whether celecoxib and nimesulide can directly repress the promoters of AR-dependent genes, reporter assays were performed using a PSA promoter-luciferase construct. As can be seen in FIG. 2, celecoxib and nimesulide significantly reduced the androgen-induced PSA promoter activity at a concentration as low as 25 μM. Because the AR binds directly to the ARE of target genes for androgen action, another reporter construct containing three tandem repeats of ARE derived from the hK2 promoter (pGL3 hK2 3xARE SV40 minimal; Mitchell et al., 2000, Prostate, 33:264-70) also was tested. The results (FIG. 2) demonstrated that both celecoxib and nimesulide significantly reduced AR/ARE-mediated gene expression (P<0.05). Thus, celecoxib and nimesulide acted as potent inhibitors of AR-mediated gene transcription.

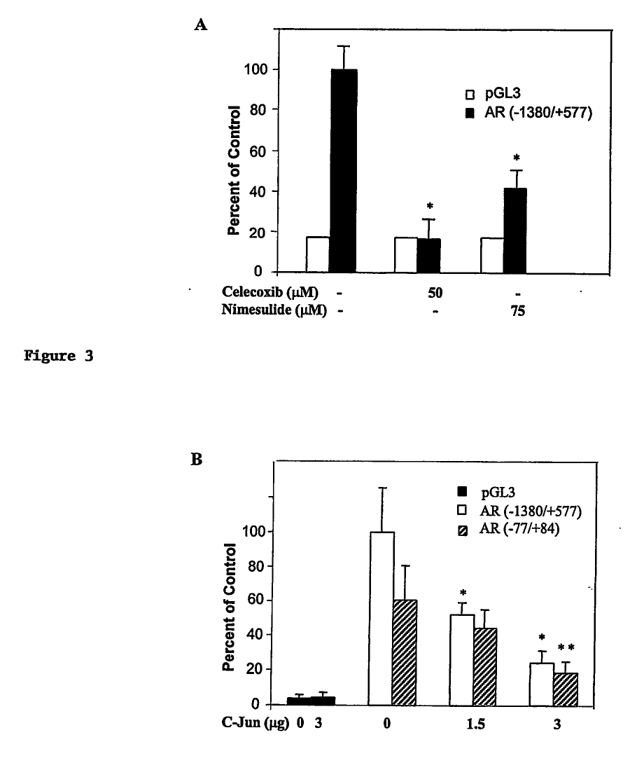

To determine whether celecoxib and nimesulide may directly affect the transcriptional activity of the AR gene, transcriptional reporter assay was performed in LNCaP cells using a luciferase reporter plasmid containing the AR promoter (−1380/+577). Compared with control vector alone, cells transfected with the AR promoter revealed significantly higher luciferase activities, as expected (FIG. 3A). However, celecoxib and nimesulide, at the concentrations used, repressed the transcription activities of the promoter (FIG. 3A). Furthermore, Western analysis using AR-specific antibody indicated that AR protein expression was reduced by celecoxib and nimesulide at concentrations used in the transfections. Taken together, these results suggest that celecoxib and nimesulide are potent inhibitors of AR function at least partially through downregulation of AR expression.

### Example 8

### Enhanced Expression and Phosphorylation of c-Jun by Celecoxib and Nimesulide in LNCaP Cells

To further dissect the molecular mechanisms underlying NSAID-mediated inhibition of AR function, the expression of c-Jun in celecoxib- and nimesulide-treated LNCaP cells was examined by Western blot analysis. Androgen-induced PSA promoter activity has been shown to be inhibited in a dose dependent manner by co-transfection with c-Jun expression plasmid. It was hypothesized, therefore, that c-Jun may potentially be involved in NSAID-mediated inhibition of AR. Results demonstrated that c-Jun protein was strongly induced by celecoxib and nimesulide at 24 h of treatment. Several previous studies have shown that the transactivation functions of the AR as well as other steroid receptors can be affected by c-Jun: Therefore, the results obtained herein strongly suggest that overexpressed c-Jun induced by celecoxib and nimesulide could interfere with AR-mediated up-regulation of PSA and hK2. It is noted that celecoxib at a relatively low concentration of 25 μM may not have an observable inhibitory effect on AR protein expression, but low concentrations of the NSAIDs could still increase c-Jun protein expression and subsequently reduced the function of the AR, as evident in the transfections shown in FIG. 2.

c-Jun is usually a short-lived protein, and it can be induced by many extracellular stimuli. In most cases, the induction is transient at early time of stimulation. However, the results described herein show that c-Jun protein levels were elevated after 15 and 24 h of treatments, implying that the NSAIDs induced a prolonged overexpression of c-Jun.

### Example 9

### Overexpression of c-Jun Inhibited the AR Promoter

To determine whether overexpression of c-Jun can affect the expression of the AR gene, c-Jun expression construct was co-transfected with the two AR promoter reporter plasmids, AR promoter (−1380/+577)-pGL3 and AR promoter (−77/+84)-pGL3, respectively, in LNCaP cells. The result shown in FIG. 3B suggests that overexpression of c-Jun significantly inhibited the activity of both tested AR promoters.

### Example 10

### IL6-Mediated Activation of the Androgen Receptor

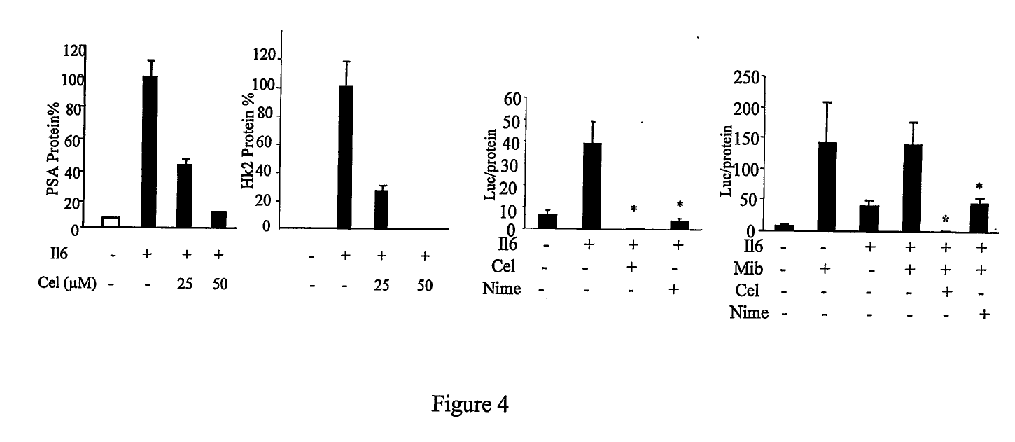

Experiments were performed to determine whether or not IL6 increased expression of PSA and/or hK2. FIG. 4A shows that IL6 (50 ng/ml) increased the amount of PSA and hK2 protein present in LNCaP cells, and that celecoxib (50 μM) significantly inhibited the IL6-induced PSA and hK2 expression in cells for 72 hrs (p<0.05). FIG. 4B shows that celecoxib (Cel; 50 μM) and nimesulide (Nime; 100 μM) inhibit androgen receptor-mediated expression of the reporter gene (hK2 ARE-SV40-pGL3) activated by IL6 (50 ng/ml) or IL6 plus androgen (1 nM) in transient transfection experiments.

Experiments were then performed to determine if STAT3 is phosphorylated following exposure to IL6. LNCaP cells were treated with IL6 (25-50 ng/ml) and celecoxib (50 μM or nimesulide (100 μM) in 5% charcoal stripped fetal calf serum and total cell extracts were prepared and used for SDS-PAGE and Western blot analysis with anti-phosphorylated STAT3 and anti-STAT3 antibodies. Consistent with previous reports, IL6 induced phosphorylation of STAT3. The phosphorylation of STAT3 by IL6 was significantly suppressed by nimesulide, but was not affected by celecoxib. It was noted that the NSAIDs did not significantly affect total STAT3 protein levels.

The same cells were treated with 50 μM of a potent phosphatase inhibitor, peroxovanadate (POV), to determine if the nimesulide-mediated inhibition of phosphorylation of STAT3 by IL6 was due to activation of one or more phosphatases. In the presence of POV, IL6 moderately enhanced the phosphorylation of STAT3 when compared to the amount of phosphorylation in the absence of POV. Therefore, the inhibitory effect of nimesulide on phosphorylation of STAT3 by IL6 may be caused by activation of one or more phosphatases, because the inhibitor could partially reverse the reduced phosphorylation of STAT3 by nimesulide. It was noted that POV alone had no effect on phosphorylation of STAT3 in the absence of IL6.

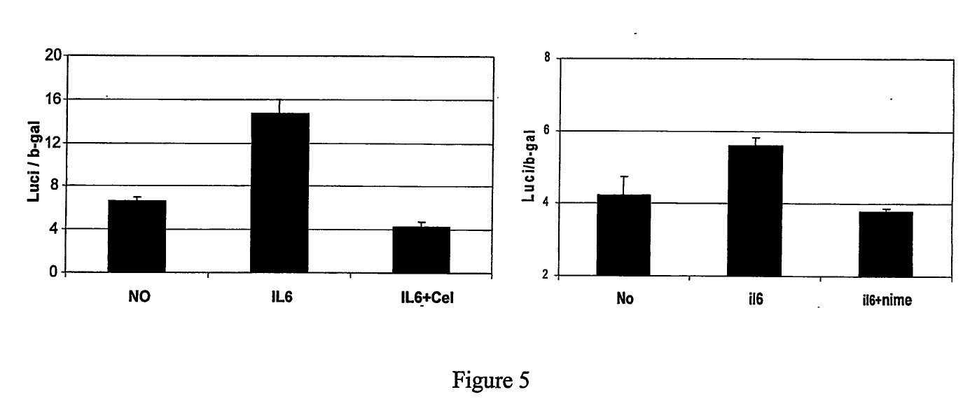

In addition, experiments were performed to determine if NSAIDs can inhibit the IL6-induced transcriptional ability of STAT3. LNCaP cells were transfected with a vector containing three STAT3 specific binding sites upstream of a minimal promoter-lucifease reporter gene and treated with IL6 (25-50 ng/ml) with or without celecoxib (cel; 50 μM) or nimesulide (nime; 100 μM) for six hours. Cell extracts were prepared for luciferase and β-gal analysis. As shown in FIG. 5, transient transfections demonstrated that the STAT3-specific responsive element-mediated luciferase expression was activated by IL6 and could be inhibited by celecoxib treatment (p<0.05). Therefore, although IL6-activated phosphorylation of STAT3 was not changed by celecoxib, it appears that the transcriptional ability of STAT3 was not active in the presence of celecoxib. The data reported herein suggests that nimesulide and celecoxib may use different mechanisms to inhibit IL6-mediated activation of the androgen receptor.

Co-immunoprecipitation assays were used to determine if an interaction between the androgen receptor and STAT3 could be detected in LNCaP cells. After exposure to 1 nM Mib, or 50 ng/ml IL6, cell extracts (400 μg proteins each reaction) were prepared from the treated LNCaP in RIPA buffer and used for immunoprecipitation with anti-androgen receptor antibody and then Protein G-coupled sepharose for pulling down the androgen receptor. The immunoprecipitates were used for SDS-PAGE and Western blotting sequentially with anti-phosphorylated STAT3, anti-STAT3, and anti-androgen receptor antibodies. Results indicated that the androgen receptor and STAT3 formed a complex independent of androgens or IL6. In the presence of IL6, phosphorylated STAT3 also formed a complex with the androgen receptor.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of monitoring the proliferation of cultured prostate cancer cells in the presence of one or more NSAIDs, comprising:
contacting the prostate cancer cells with one or more NSAIDs; and
determining the level of expression, the transactivating ability, and/or the IL6-mediated activation of an androgen receptor,
wherein a decrease in the expression, the transactivating ability, and/or the IL6-mediated activation of the androgen receptor indicates an inhibitory effect by the NSAID on the proliferation of the prostate cancer cells.

2. The method of claim 1, wherein the one or more NSAIDs is celecoxib and/or nimesulide.

3. A method of treating an individual with prostate cancer or at risk of developing prostate cancer, comprising:
identifying an individual with prostate cancer or at risk of developing prostate cancer; and
administering a dose of one or more NSAID to the individual in an amount effective to inhibit expression, transactivating ability, and/or IL6-mediated activation of an androgen receptor,
wherein an inhibition of the expression, the transactivating ability, and/or the IL6-mediated activation of the androgen receptor inhibits the proliferation of prostate cancer cells, thereby treating the individual.

4. The method of claim 3, wherein the effective dose is from about 10 mg/kg to about 300 mg/kg.

5. The method of claim 3, wherein the one or more NSAIDs is celecoxib and/or nimesulide.

6. The method of claim 3, wherein the individual is human.

7. The method of claim 3, wherein the administration is selected from the group consisting of orally, transdermally, intravenously, intraperitoneally, or using an implant.

8. The method of claim 3, further comprising:
monitoring the expression, the transactivating ability, and/or the IL6-mediated activation of the androgen receptor in the individual.

9. The method of claim 3, further comprising:
monitoring the individual for a dose-dependent reduction in prostate-specific antigen (PSA) levels,
wherein a dose-dependent reduction in PSA correlates with a dose-dependent decrease in the expression, the transactivating ability, and/or the IL6-mediated activation of the androgen receptor.

10. The method of claim 9, further comprising:
adjusting, if necessary, the dose of the one or more NSAIDs to achieve or maintain the dose-dependent reduction in PSA.

11. The method of claim 3, further comprising:
monitoring the individual for a reduction in human glandular kallikrein (hK2) levels, wherein a reduction in hK2 correlates with a decrease in the expression, the transactivating ability, and/or the IL6-mediated activation of the androgen receptor.

12. The method of claim 11, further comprising:
adjusting, if necessary, the does of the one or more NSAIDs to achieve or maintain the reduction in hK2.

13. A method of reducing the risk of recurrence of prostate cancer in an individual, wherein the individual previously had been treated for prostate cancer, comprising:
administering a dose of one or more NSAIDs to the individual in an amount effective to inhibit expression, transactivating ability, and/or IL6-mediated activation of an androgen receptor,
wherein inhibiting the expression, the transactivating ability, and/or the IL6-mediated activation of the androgen receptor inhibits the proliferation of prostate cancer cells, thereby reducing the risk of recurrence of prostate cancer in the individual.

14. The method of claim 13, wherein the one or more NSAIDs is celecoxib and/or nimesulide.

15. The method of claim 13, further comprising:
monitoring the expression, the transactivating ability, and/or the IL6-mediated activation of the androgen receptor in the individual.

16. The method of claim 13, further comprising:
monitoring the individual for a dose-dependent reduction in prostate-specific antigen (PSA) levels,
wherein a dose-dependent reduction in PSA correlates with a dose-dependent decrease in the expression, the transactivating ability, and/or the IL6-mediated activation of the androgen receptor.

17. The method of claim 16, further comprising:
adjusting, if necessary, the dose of the one or more NSAIDs to achieve or maintain the dose-dependent reduction in PSA.

18. The method of claim 13, further comprising:
monitoring the individual for a reduction in human glandular kallikrein (hK2) levels, wherein a reduction in hK2 correlates with a decrease in the expression, the transactivating ability, and/or the IL6-mediated activation of the androgen receptor.

19. The method of claim 18, further comprising:
adjusting, if necessary, the does of the one or more NSAIDs to achieve or maintain the reduction in hK2.

20. The method of claim 13, wherein the previous treatment for prostate cancer in the individual comprised a radical prostectomy.

21. A method of treating an individual with benign prostatic hyperplasia (BPH) or at risk of developing BPH, comprising:
identifying an individual with BPH; and
administering a dose of one or more NSAIDs to the individual in an amount effective to inhibit expression, transactivating ability, and/or IL6-mediated activation of an androgen receptor,
thereby treating the individual.

22. The method of claim 21, wherein the one or more NSAIDs is celecoxib and/or nimesulide.

23. The method of claim 21, further comprising:
monitoring the expression, the transactivating ability, and/or the IL6-mediated activation of the androgen receptor in the individual.

24. The method of claim 21, further comprising:
monitoring the individual for a dose-dependent reduction in prostate-specific antigen (PSA) levels,
wherein a dose-dependent reduction in PSA correlates with a dose-dependent decrease in the expression, the transactivating ability, and/or the IL6-mediated activation of the androgen receptor.

25. The method of claim 24, further comprising:
adjusting, if necessary, the dose of the one or more NSAIDs to achieve or maintain the dose-dependent reduction in PSA.

26. The method of claim 21, further comprising:
monitoring the individual for a reduction in human glandular kallikrein (hK2) levels, wherein a reduction in hK2 correlates with a decrease in the expression, the transactivating ability, and/or the IL6-mediated activation of the androgen receptor.

27. The method of claim 26, further comprising:
adjusting, if necessary, the does of the one or more NSAIDs to achieve or maintain the reduction in hK2.

28. A method of screening for compounds that inhibit the proliferation of prostate cancer cells, comprising:
contacting prostate cancer cells with a compound; and
determining the level of expression, the transactivating ability, and/or the IL6-mediated activation of an androgen receptor,
wherein decreased expression, transactivating ability, and/or IL6-mediated activation of the androgen receptor in the prostate cancer cells compared to prostate cancer cells not contacted with the compound indicates a compound that inhibits the proliferation of prostate cancer cells.

29. The method of claim 28, wherein the one or more NSAIDs is celecoxib and/or nimesulide.

30. A composition comprising:
one or more NSAIDs;
one or more compounds that has a mechanism of action selected from the group consisting of:
inhibiting expression of a gene encoding an androgen receptor,
inhibiting nuclear localization of an androgen receptor, and
inhibiting the transactivating ability of an androgen receptor; and
a pharmaceutically acceptable carrier.

31. The composition of claim 30, wherein the one or more NSAIDs is celecoxib and/or nimesulide.

32. The composition of claim 30 wherein the compound is selected from the group consisting of silymarin, silibin, docosahexaenoic acid (DHA), eicosapentaenoic acid (EPA), quercetin, perillyl alcohol (POH) or a derivative thereof, resveratrol, flufenamic acid, tea polyphenols, and anti-androgen compounds.

33. An article of manufacture comprising the composition of claim 30; and packaging material, wherein the packaging material comprises instructions for using the composition to inhibit expression, transactivating ability, and/or IL6-mediated activation of an androgen receptor in an individual.

34. The article of manufacture of claim 33, further comprising:
compositions for monitoring the expression, the transactivation, and/or the IL6-mediated activation of the androgen receptor.

35. The article of manufacture of claim 33, further comprising:
compositions for monitoring PSA.

36. The article of manufacture of claim 33, further comprising:
compositions for monitoring hK2.

37. A composition comprising one or more NSAIDs, wherein the one or more NSAIDs are formulated for transdermal delivery to the prostate of an individual, wherein delivery to the prostate inhibits the expression, the transactivating ability, and/or the IL6-mediated activation of the androgen receptor.

38. The composition of claim 37, wherein the one or more NSAIDs is celecoxib and/or nimesulide.

39. A composition comprising one or more NSAIDs, wherein one or more NSAIDs are formulated for implantation near the prostate of an individual, wherein implantation near the prostate inhibits the expression, the transactivating ability, and/or the IL6-mediated activation of the androgen receptor.

40. The composition of claim 39, wherein the one or more NSAIDs is celecoxib and/or nimesulide.
